(19) United States
(12) Patent Application Publication
(10) Pub. No.: US 2011/0105948 A1
(43) Pub. Date: May 5, 2011

(54) Electrical Impedance Sensing Biopsy Sampling Device And Method

(75) Inventor: Ryan Joseph Halter, Orford, NH (US)

(73) Assignee: The Trustees of Dartmouth College a Nonprofit Corporation of Higher

(21) Appl. No.: 12/994,055

(22) PCT Filed: May 7, 2009
(86) PCT No.: PCT/US2009/043181

§ 371 Date: Nov. 22, 2010

Related U.S. Application Data
(60) Provisional application No. 61/055,685, filed on May 23, 2008.

(51) Int. Cl. A61B 10/02

(52) U.S. Cl. 600/567

(57) ABSTRACT

A biopsy sampling device has an inner trocar having a sharpened tip and a sampling opening. The trocar is slideably engaged in a central cavity of an outer needle with its sharpened tip and sampling opening protruding from an end of the outer needle. Impedance measuring apparatus measures impedance between the inner trocar and the outer needle. In an embodiment, impedance is measured at multiple frequencies and spectral characteristics are determined therefrom. The inner trocar has an insulating coating over those portions of the inner trocar that are located within the central cavity of the outer needle. In an embodiment, the sampling device is used to obtain samples from an organ at points in the organ where impedance differs from an impedance of organ stroma.

### RELATED APPLICATIONS

The present application claims priority to U.S. provisional patent application No. 61/055,685, filed May 23, 2008, incorporated herein by reference.

### GOVERNMENT RIGHTS

The present apparatus was developed with the aid US Department of Defense Congressionally Directed Medical Research Program grant W81XH-07-1-0104. The United States Government has certain rights in the herein described apparatus.

### FIELD

The present apparatus relates to the field of tissue sampling devices for obtaining specimens of tissue for pathological examination, and to the field of medical instrumentation devices.

### BACKGROUND

When lumps, tumors, inhomogenicities, or other inclusions appear within human and other biological tissues, it is often desirable to obtain samples of the inclusion for analysis so as to determine a type inclusion. Typing of inclusions is desirable to assist in determining an appropriate treatment, or whether treatment is necessary; since some inclusions may be malignant, others benign, and others may be abscesses or cysts. Cysts and abscesses require quite different treatment from malignant inclusions.

Samples are often taken from inclusions using a sampling device having an outer tube and an inner probe or needle having a cutting cavity on a side. The device is inserted into the inclusion and the inner probe or needle is operated to capture a small piece of tissue from the inclusion in the cavity. The device is removed and the sample analyzed.

A problem when taking samples of inclusions, especially smaller inclusions, in tissues is that it can be difficult to ensure that the sample is taken of the inclusion and not of adjacent, likely healthy, tissue. When normal, nearby, tissue is sampled instead of the inclusion, pathological analysis of the sample will not give a correct diagnosis and may give sufficiently misleading information that no or inappropriate treatment is provided to patients instead of appropriate curative treatment.

In order to obtain samples from an inclusion instead of from normal tissue, imaging-guided biopsy techniques may be used. For example, Computed Tomography (CT)—guided biopsy techniques are often used with some organs. These techniques require taking multiple images of a patient to observe both the inclusion and a sampling device; the images are taken at intervals during the process of inserting and manipulating the sampling device into the inclusion. CT-guided biopsy techniques pose issues with high radiation dose from multiple CT images, and do not always provide good resolution of the inclusions especially when the inclusions are in low density tissues surrounded by high density tissues. Further, CT machines are somewhat bulky and moderately expensive.

WO/2002/085216 describes a biopsy sampling device adapted for Magnetic Resonance Imaging (MRI)-guided biopsies. This device has an outer shield and an inner probe, where the inner probe is electrically insulated from the outer shield by an insulation layer on the inner conductor. This device serves as a radio-frequency antenna to sense resonance during operation of an MRI system, requiring an expensive MRI machine during taking of a biopsy sample. Also, since it is intended for use within the intense magnetic field of an MRI system, it must be made of non-ferrous exotic materials such as gold that are not affected by, and do not affect, the magnetic field of the MRI machine. This device is used to sample an organ while imaging the organ, so that samples may be obtained from particular suspicious inclusions within the organ.

It is desirable to find alternative ways of guiding biopsy sampling devices to obtain samples of tumors and other inclusions in organs; in particular it is desirable to find ways that do not require use of such an expensive and bulky device as an MRI imaging system while obtaining biopsy samples for pathological analysis. It is also desirable to sense the pathological state of the tissue in area close to where the sample was collected to provide a more accurate estimate of disease extent, if any.

### SUMMARY

A biopsy sampling device has an inner trocar having a sharpened tip and a sampling opening. The trocar is slideably engaged in a central cavity of an outer needle with its sharpened tip and sampling opening protruding from an end of the outer needle. Electrical impedance measuring apparatus measures electrical impedance between the inner trocar and the outer needle. The inner trocar has an insulating coating over those portions of the inner trocar that are located within the central cavity of the outer needle.

In an embodiment, impedance measurements are made at multiple frequencies and spectral parameters are obtained from the measurements.

In an embodiment, the sampling device is used to obtain samples from an organ at points in the organ where impedance differs from an impedance of normal or surrounding organ tissue.

In an embodiment, the sampling device is used to obtain impedance measurements from an organ at points in the organ where biopsy samples are taken, these are points where impedance has potential to differ from normal or surrounding organ tissue.

The impedance measurements are used together with pathological analysis of the samples to formulate a diagnosis and treatment plan.

### BRIEF DESCRIPTION OF THE DRAWINGS

FIG. 1 is a sectional diagram of a tip of a biopsy sampling device.

FIG. 2 is a block diagram of a biopsy sampling device.

FIG. 3 is a flow chart of one method of operating the sampling device wherein the device is used to obtain impedance information at sample points within an organ.

FIG. 4 is a flow chart of a method of operating the sampling device wherein the device is used to guide acquisition of samples while advancing the sampling device to desired sample locations.

### DETAILED DESCRIPTION OF THE EMBODIMENTS

In a study of radical prostatectomy specimens (Halter R J, Schned A R, Heaney J A, et al. Electrical impedance spectroscopy of benign and malignant prostatic tissues. Journal of Urology, 179(4):1580-1586, 2008) from fourteen men, it was found that some tumors of the prostrate have an electrical impedance (inverse of admittance) that differs from the electrical properties of surrounding, normal, tissues. In particular, at least some adenocarcinoma (malignant) tumors of the prostate were found to have electrical conductivity and permittivity (components of electrical impedance) that differed from tissues associated with benign prostate hypertrophy or normal prostate stroma at frequencies of greater than 92 KHz. Particular samples of adenocarcinoma of the prostate were found to have significantly lower conductivity (higher resistance) than normal prostate stroma.

A more recent study Halter R J, Schned A R, Heaney J A, et al. Electrical properties of prostatic tissues: I. Single frequency admittivity properties, accepted for publication sometime in 2009 in Journal of Urology has also been done. In this study of tissue samples of adenocarcinoma, benign prostatic hyperplasia, non-hyperplastic glandular tissue, and stroma samples taken from radical prostatectomy specimens from 50 men, it was shown that, in addition to significant conductivity differences between malignant and benign prostate tissue, there are significant permittivity differences. The direction and magnitude of these differences changes depending on the frequency at which the electrical properties were gauged. In particular, the permittivity of prostate cancer at 100 kHz is twice that of benign prostatic hyperplasia, non-hyperplastic glandular tissue, and normal prostatic stroma. When permittivity at 100 kHz was used to discriminate cancer from benign tissues it provided a specificity of 77% at a sensitivity level of 70%.

The electrical properties of tissue are a function of the AC frequency at which they are sampled. This frequency dependence is also a function of tissue morphology and this spectral dependence has the potential to provide enhanced clinical utility. In this same cohort of 50 men, the electrical properties were sampled at 31 logarithmically spaced frequencies ranging from 100 Hz to 100 kHz. Four multi-frequency based spectral parameters defining the recorded spectrum (σ_{∞}, Δσ, f_{c}, and α) using a Cole-type model were extracted from each of the electrical property spectra. The Cole-type model is similar to that described in Cole K S and Cole R H,

Dispersion and absorption in dielectrics: I. Alternating current characteristics J Chem Phys, 9: 341-351, 1941. These spectral parameters are typically thought to represent:

1) σ_{∞} (extrapolated impedance at infinite frequency): a measure of cumulative intra- and extra-cellular fluid conductivity

2) Δσ (difference between extrapolated impedance at zero frequency and at infinite frequency): a measure of the intra- and extra-cellular volume

3) f (a relaxation frequency, derived as an inverse of a relaxation time): a measure of cell membrane quantity and viability

4) α (a measure of a broadness of the spectra): a measure of tissue heterogeneity

The results of the spectral decomposition are presented in Halter R J, Schned A R, Heaney J A, et al. Electrical properties of prostatic tissues: II. Spectral admittivity properties, accepted for publication sometime in 2009 in Journal of Urology. Significant differences between malignant and benign prostate were noted for σ_{∞}, Δσ, and f_{c}. Of the spectral parameters, f_{c} provided the best cancer discrimination with a specificity of 81.5% at a sensitivity level of 70%. Spectral representations other than the Cole-model can be employed to parameterize the frequency-dependent electrical properties. These spectral parameters provide more contrast than the discrete frequency parameters (conductivity and permittivity), but require a longer acquisition time since the electrical properties at multiple frequencies must be sampled. Depending on the clinical situation either spectral or discrete frequency electrical properties could be gauged.

Finally, these electrical properties (both discrete frequency and spectral) provide enhanced discriminatory power when just high-grade prostate cancers were compared to all benign tissues.

It is believed that electrical conductivity and permittivity measurements, and the Cole-Cole spectral parameters derived from them, made using a biopsy sampling device as an electrode will provide information about tissue near the sampling device at the time a sample is taken, and may be able to provide some guidance to a physician so that samples may be taken of malignant inclusions as well as surrounding tissues.

These conductivity and permittivity measurements may also provide some additional diagnostic information regarding the extent of disease since many biopsy samples only show a small foci of cancer. These measurements will indicate if the tissue surrounding the biopsy site is diseased or not.

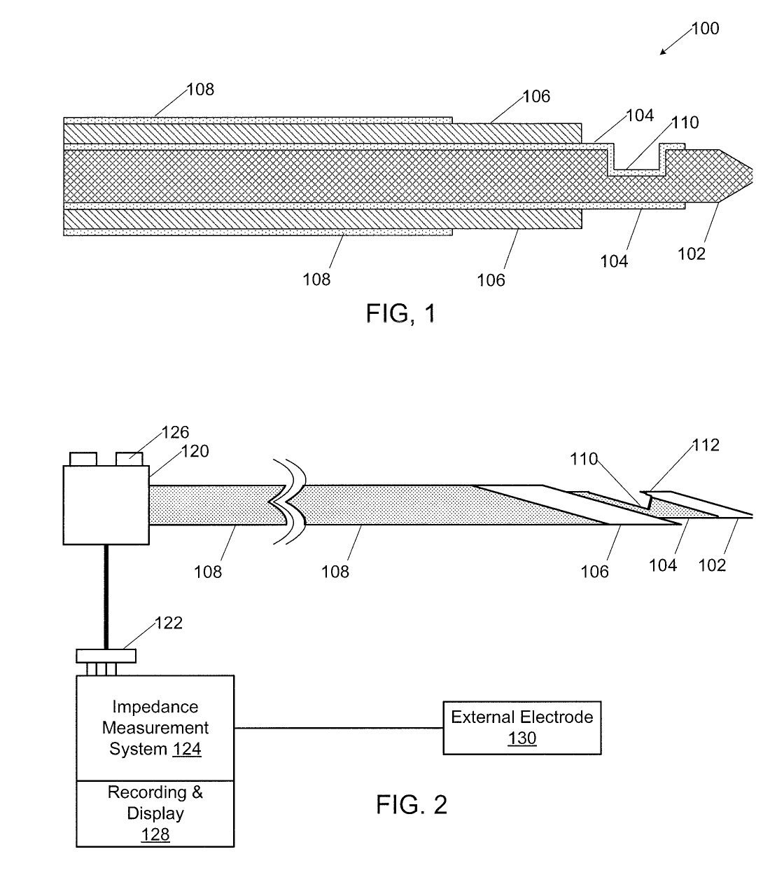

A biopsy sampling device 100 (FIGS. 1 and 2) has a central sampling needle trocar 102, which may have a single-tapered (as shown in FIG. 2) or a double-tapered (as shown in FIG. 1) sharpened tip. The central sampling needle trocar 102 has an electrically insulating, biocompatible, coating 104 adherent thereto, such as Epoxylite® 6000 M. Epoxylite is a trademark of Elantus PDG Inc, St Lois, Mo., a subsidiary of Elantas of Wessel, Germany.

The central sampling needle trocar 102 and it's coating 104 is slideably engaged within an outer hollow needle 106. In an embodiment, outer hollow needle 106 is an 18-gauge needle. Similarly, all but a tip portion of outer hollow needle 106 is coated with an outer-needle insulating, biocompatible, coating 108 adherent thereto; in an embodiment this is formed of the same Epoxylite 6000 M material used for the coating on the sampling needle trocar 102. In an embodiment, the uninsulated tip portion of the outer needle has length about two millimeters.

In an embodiment, insulating coating 104 is less than fifty microns thick so that the central sampling needle trocar 102 of about ninety-nine hundredths inch diameter can freely slide within the outer hollow needle 106.

Since the sampling device 100 need not be used in a magnetic resonance imaging environment, in an embodiment the central sampling needle trocar 102 and outer hollow needle 106 are made of ferrous metal, such as stainless steel as known in the surgical instrument art.

Central sampling needle trocar 102 has a sample slot 110 cut into it. When the device is inserted into tissue with the sampling needle trocar fully extended, tissue—possibly including a portion of an inclusion—enters the sample slot 110. The sampling needle trocar 102 may then be withdrawn through the outer needle 106 and a cutting edge 112 separates a sample of the tissue from the tissue. The sample may be placed in a pathology sample container (not shown) and the sampling needle trocar 102 reinserted into the outer needle 106 to obtain additional samples.

Outer hollow needle 106 is fitted with a manipulation handle 120, which is adapted with mechanical keying apparatus such that, in embodiments like that of FIG. 2 with a single-tapered tip, sampling needle trocar 102 is not free to rotate with respect to outer hollow needle 106. Four wires are brought out to a connector 122 from the needles 102, 106, two attached to the sampling needle trocar 102 and two to the outer needle 106. One wire attached to sampling needle trocar 102 is coupled through connector 122 to a stimulus circuit of impedance measuring system 124, the other wire connected to sampling needle trocar 102 is coupled to a measurement circuit of impedance measuring system 124. Similarly, one wire attached to outer needle 106 is coupled through connector 122 to a stimulus circuit of impedance measuring system 124, the other wire connected to outer needle 106 is coupled to a measurement circuit of impedance measuring system 124 having display and recording apparatus 128. In an alternative embodiment, more subject to interference by dirty or loose connections, only two electrical connections are used, one to the trocar 102 and one to the outer needle 106. In an alternative embodiment, there is also one or more external electrodes 130 for contacting a surface of a subject.

The manipulation handle 120 is also fitted with an impedance test button 126 to trigger measurement and acquisition of electrical impedance data.

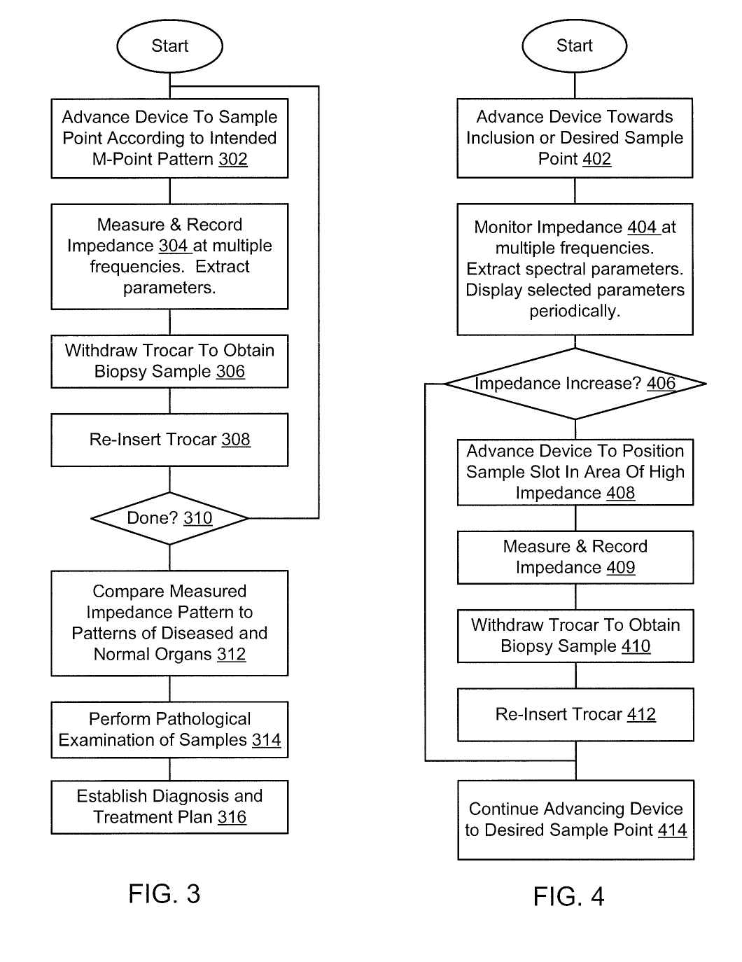

In a first method of operation, as illustrated in FIG. 3, the tip of the sampling device is advanced 302 into the prostate, or into another organ of the subject that is suspected of having an inclusion requiring biopsy, to a point at which it is desired to obtain a sample. Guidance of the sampling device may be according to a predetermined pattern or according to images obtained from a separate imaging modality. Once at the sampling point, the impedance test button 126 is pressed; at this time high frequency impedance characteristics of the tissue are measured 304 by applying a low current, high frequency, stimulus current having at least one, and in an embodiment several, frequencies between 100 Hz and 1 MHz by stimulus circuits of the impedance measuring system 124, and measuring voltages developed between the needles with the measurement circuit of impedance measuring system 124, these measurements are recorded. In the alternative embodiment having an external electrode 130, impedance measurements may also be taken between either needle 106 or trocar 102 and the external electrode 130.

Four multi-frequency based spectral parameters defining the recorded spectrum (σ_{∞}, Δσ, f_{c}, and α) using the Cole-type model are then extracted from the recorded impedance measurements. The sampling needle trocar 102 is then withdrawn 306 to excise and remove a sample from the organ for pathological analysis. Since the stimulus current flows through a radius of about 2½ millimeters around the tip of sampling device 102 several cubic millimeters of the organ are sampled. The measured conductivity, permittivity, and spectral impedance properties give information not just of the sample, but of a region near the sample that may or may not contain possible tumors. If 310 all desired samples have not yet been taken, the trocar 102 is reinserted 308 and the sampling device tip advanced further or otherwise repositioned to obtain additional samples; as an example additional samples might be collected following a predetermined, 12-point, pattern as is often used for prostate biopsy.

Once 310 all desired samples have been taken, the measured pattern of conductivity, permittivity, and spectral parameters, measured within the organ is compared 312 to patterns of conductivity, permittivity, and spectral parameters of both normal and diseased organs. Pathological examination of samples is also performed 314. Both information from the pattern of impedance and spectral parameters, and from the pathological examinations are used to establish 316 a diagnosis and treatment plan. In this method, the impedance and spectral parameter measurements give additional information about tissue characteristics surrounding an analyzed sample that is useful for diagnosis 316, and in particular useful for estimating tumor size and aggressiveness.

The estimated tumor size and aggressiveness is critical to tumor staging; tumor staging in turn is of great interest in devising a treatment plan. In particular, large rapidly growing tumors may require radical prostatectomy, while smaller tumors are more likely to be treated by less invasive techniques such as transurethral resection or active surveillance.

In an alternative method of operation, as illustrated in FIG. 4, impedance changes are used to guide sampling while advancing the sampling device into an organ 302 (FIG. 3) along a path guided by other imaging modalities towards an inclusion or a path otherwise aimed towards a desired sample location. In this method, the impedance characteristics of the tissue are monitored 404 by applying a high frequency stimulus current from the stimulus circuits of the impedance measuring system 124 and measuring voltages developed between the needle 106 and trocar 102, or in an alternative embodiment between trocar 102 and external electrode 130, with the measurement circuit of impedance measuring system 124, the measurements are averaged and filtered over a short period of time to avoid artifacts, the spectral parameters are extracted, and selected measurements are displayed to a operator. When the operator sees a change of impedance in a suspect direction, such as an increase of impedance 406, indicating a suspect inclusion is within a few millimeters of the tip of the sampling device 100, the operator advances 408 the sampling device 100 a predetermined distance of a few millimeters until the suspect inclusion is expected to be nearest to the sample slot 110.

Once the sampling device is positioned within the area of suspect impedance, impedance is measured 409 and recorded, and the center trocar 102 of the sampling device is then withdrawn 410 to obtain a biopsy sample of the suspected inclusion. Once the sample is placed in a sample container, the center trocar 102 is reinserted 412 into the sampling device and advancement sampling device is then continued towards other locations, such as predetermined locations or locations guided by other imaging methods, within the organ from which samples are to be taken.

Therefore, both samples according to predetermined locations in the organ and samples according to impedance changes may be taken and submitted for pathological analysis for diagnostic purposes. Information from pathological analysis of the samples, and information from comparing a measured pattern of impedance and spectral parameters at the sampling points to known impedance patterns and spectral parameters of normal and diseased organs, are used in establishing a diagnosis 316 and treatment plan.

In an alternative embodiment of the method, after positioning the sampling slot 110 of the trocar 102 of the device 100 by advancing it into an area of interest in the organ, the outer needle 106 is advanced to excise a sample. The trocar 102 is then removed to transfer the sample to a pathology sample container and reinserted into the outer needle 106 before advancing the device to any additional sampling points.

It is expected that the electrical impedance measurement and monitoring described herein can be added to other biopsy sampling devices that may be known in the art of Medicine

While the forgoing has been particularly shown and described with reference to particular embodiments thereof, it will be understood by those skilled in the art that various other changes in the form and details may be made without departing from the spirit hereof. It is to be understood that various changes may be made in adapting the description to different embodiments without departing from the broader concepts disclosed herein and comprehended by the claims that follow.

What is claimed is:

1. A biopsy sampling device comprising:
an inner trocar having a sharpened tip and a sampling opening;
an outer needle having a central cavity;
an impedance measuring apparatus coupled to measure an impedance between the inner trocar and the outer needle;
the inner trocar slideably engaged within the central cavity of the outer needle such that its sharpened tip and sampling opening protrude from an end of the outer needle;
wherein the inner trocar has an insulating coating over those portions of the inner trocar that are located within the central cavity of the outer needle;
and wherein the inner trocar is adapted to be removed from the outer needle, thereby capturing a sample in the sampling opening.

2. The biopsy sampling device of claim 1 wherein the outer needle is insulated except for an uninsulated portion near the end from which the inner trocar protrudes.

3. The biopsy sampling device of claim 2 wherein the uninsulated portion near the end of the outer needle has a length of two millimeters.

4. The biopsy sampling device of claim 1 wherein the inner trocar is made of ferrous metal.

5. The biopsy sampling device of claim 1 further comprising impedance measurement apparatus for measuring an alternating current impedance at at least one frequency between one hundred and one million hertz, the impedance measurement apparatus coupled to at least one of the needle and trocar.

6. The biopsy sampling device of claim 5 further comprising an external electrode.

7. The biopsy sampling device of claim 1 wherein the impedance measurement apparatus measures alternating current impedance at several frequencies between one hundred and one hundred thousand hertz, and computes spectral parameters from the measurements, and displays at least one spectral parameter to a user.

8. A method of obtaining biopsy samples from a subject comprising:
advancing a sampling device into an organ while monitoring impedance characteristics between a center trocar of the sampling device and an electrode selected from the group consisting of an outer needle of the sampling device and an external electrode;
displaying to an operator at least one impedance characteristic, the impedance characteristic updated regularly;
upon observing a change of impedance, withdrawing the center trocar of the sampling device to obtain a biopsy sample of an inclusion in the organ.

9. The method of claim 8 wherein the electrode selected from the group consisting of an outer needle of the sampling device and an external electrode is an outer needle of the sampling device.

10. The method of claim 8 wherein the at least one impedance characteristic displayed to an operator includes at least one spectral parameter.

11. The method of claim 10 further comprising advancing the sampling device past a point where the change of impedance was observed such that a sampling opening of the center trocar is positioned within the inclusion in the organ.

12. A method of diagnosing inclusions within an organ within a subject comprising:
advancing a sampling device into the organ while monitoring impedance characteristics between a center trocar of the sampling device and an outer needle of the sampling device;
displaying to an operator the impedance characteristics;
upon observing a change of impedance in the displayed impedance, positioning the sampling device such that a sampling opening of the sampling device is positioned to capture a biopsy sample of the inclusion;
removing the biopsy sample from the organ; and
performing pathological examination of the biopsy sample.

13. The method of claim 12 wherein the at least one impedance characteristic displayed to an operator includes at least one spectral parameter.

14. The method of claim 13 further comprising:
obtaining impedance measurements and biopsy samples from a plurality of locations within the organ; and
using both the impedance measurements and pathological examination of the biopsy samples to determine the diagnosis.

15. A biopsy sampling device comprising:
a first conductive part having a sampling opening;
a second conductive part adapted to cover the sampling opening;
an impedance measuring apparatus coupled to measure an impedance between the first part and the second part;
wherein a part selected from the first part and the second part has an insulating coating over those portions of the part that may contact another part during insertion of the sampling device into an organ.
